(19) United States
(12) Patent Application Publication
(10) Pub. No.: US 2015/0025332 A1
(43) Pub. Date: Jan. 22, 2015

(54) BODY BALANCE CORRECTION SYSTEM

(71) Applicant: Heewoon Yang, Seoul (KR)

(72) Inventor: Heewoon Yang, Seoul (KR)

(21) Appl. No.: 14/370,523

(22) PCT Filed: Jul. 29, 2011
(86) PCT No.: PCT/KR2011/005602

§ 371 Date: Jul. 3, 2014

(30) Foreign Application Priority Data
Jul. 30, 2010 (KR) 10-2010-0073996

(51) Int. Cl. A61B 5/11; A61B 5/00; A61B 5/01; G05B 15/02; A61B 5/103

(52) U.S. Cl. CPC A61B 5/1121; G05B 15/02; A61B 5/1036; A61B 5/01; A61B 5/7246; A61B 5/742
USPC 600/301; 600/587

(57) ABSTRACT

There is provided a body balance correction system and a method for controlling same, wherein body balance of a user is allowed to be improved in his or her daily life. The body balance correction system includes a measurement unit which measures data on the left and right sides of an examinee's body, a control unit which analyzes the data measured by the measurement unit and provides a selection criterion for a body balance correction product based on a result of the analyzed data, and a display unit which displays the result of the analyzed data.

### TECHNICAL FIELD

The present invention a body balance correction system and a method for controlling same, specifically wherein body balance is improved in daily life.

### BACKGROUND ART

In general, body imbalance, that is, postural imbalance in daily life may cause spinal deformity and impose strain on the musculoskeletal system, end thus diseases may occur. A variety of health care methods and medical technologies have been developed to solve the body imbalance. However, the conventional health care methods and medical technologies for health management usually remain at the level of partial medical care or treatments. Accordingly, although temporarily useful, the conventional health care or medical treatments may fail to be a fundamental solution to the body imbalance. Consequently, repeated health care or medical treatments are required to provide continuous health management, but this has a disadvantage that requires a large amount of time, costs, and efforts.

### DISCLOSURES

### Technical Problem

The present invention is applied to solve the defects, and an objective of the present invention is to provide a body balance correction system and a method for controlling same which obtain and analyze data on body balance, and then provide a body balance correction product that corrects body balance based on a result of the obtained and analyzed data, thereby allowing a user to improve his or her body balance in daily life after the user wears the body balance correction product.

### Technical Solution

To achieve the objective of the present invention, a body correction system for diagnosing and correcting the balanced state of a user's body includes a measurement unit which resources data on the left and right sides of an examinees's body, a control unit which analyzes the data reasoned by the measurement unit and provides a selection criterion for a body balance correction product based on a result of the analyzed data, and a display unit which displays the result of the analyzed data. The measurement unit may include a pressure sensor which senses body pressure of the left, right, front, and rear parts of the examinee's body, and a thermal sensor which senses temperatures of different parts of the examinee's left and right foot soles.

The measurement unit may further include a foot size measurement unit for measuring the size of the examinee's foot. The pressure sensor has a plurality of pressure sensors which sense body pressure of the left, right, front, and rear parts of the examinee's body, and the control unit senses the degree by which the examinee's body is balanced, on the basis of the pressure data measured by a plurality of the pressure sensors.

The control unit analyses the body data measured by the measurement unit, based on at least one of a correlation between the foot skeleton, and the spine, the cervical spine, and the pelvis, another correlation between, the spine and body pressure, and a further correlation between the human organs and the feet.

The control unit calculates at least one of strength, height, and a momentum adjustment value of a balance correction member configuring the body balance correction product so that the analyzed result is modified according to a predetermined reference value, and then provides a selection criterion.

In addition, the present invention may further include a storage unit which stores the data measured by the measurement unit as well as the result analyzed by the control unit.

The display unit displays at least one of the following data on the examinee: personal information; examination date; a history of examinations; a pressure distribution on the left and right sides of the body; a temperature distribution on the left and right foot soles; a body balance state of the examinee when an examination is performed; a correlation between the foot skeleton and the spine; a correlation between body pressure and the spine; and a correlation between the human organs and the feet.

The display unit displays an analyzed result of how the human organs are distributed on foot reflex zones, based on a temperature distribution on any one of the left and right foot soles, the foot reflex zones shoving a correlation between the human organs and the feet, and unusual points of the temperature distribution.

The display unit displays a temperature distribution on at least one of the left and right foot soles, a spine diagram shoving a correlation between the spine, the pelvis, and the feet, and unusual points the temperature distribution, and then shows an analyzed result of twisted portions or degrees of the spine and the pelvis on the spine diagram.

The display unit uses a history of the examinee's examinations that are performed many times to compare and display the examination results measured at times different from each other.

Here, the body balance correction product may be a pair of cushioning shoes which includes an outsole, a midsole that is disposed over the outsole, a plurality of cushioning members that are interposed between the midsole and the outsole to locally reduce impacts, and an insole that is coupled to the midsole and accommodates a user's foot.

In addition, to achieve the above-mentioned objective, a method for controlling a body balance correction system that diagnoses and corrects body balance includes measuring data on the left and right sides of an examinee's body, analyzing the data measured by the measurement unit, providing a selection criterion for a body balance correction product based on a result of the analyzed data, and displaying the result of the analyzed data.

The measuring data on the left and right sides of the examinee's body includes sensing body pressure of the left, right, front, and rear parts of the examinee's body, sensing temperatures of different parts of the examinee's left and right foot soles, and measuring the sire of the examinee's foot.

Also, the analyzing the data measured by the measurement unit may further include analyzing the degree by which the examinee's body is balanced when measuring data on the examinee's body is performed.

In the analyzing the data measured by the measurement unit, the body data is analyzed, based on at least one of a correlation between the foot skeleton and the spine, the cervical spine, and the pelvis, another correlation between the spine and body pressure, and a further correlation between the human organs and the feet.

In the providing a selection criterion for a body balance correction product, at least one of strength, height, and a momentum adjustment value or a balance correction member configuring the body balance correction product is calculated so that the analyzed result is modified according to a predetermined reference value, and then a selection criterion is provided.

In addition, the method for controlling a body balance correction system according to the present invention may further include storing the data measured by the measurement unit as well as the result analyzed by the control unit.

In the displaying the result of the analyzed data, at least one of the following data on the examinee: personal information; examination date; a history of examinations; a pressure distribution on the left and right sides of the body; a temperature distribution on the left and right foot soles; a body balance state of the examinee when an examination is performed; a correlation between the foot skeleton and the spine; a correlation between body pressure and the spine; and a correlation between the human organs and the feet is displayed.

In the displaying the result of the analyzed data, examination results measured at times different from each other are compared with each other and displayed on the basis of a history of the examinee's examinations that are performed many times.

### Advantageous Effects

A body balance correction system and a method for controlling same according to the present invention obtain and analyze data on body balance, and then provide a body balance correction product that corrects body balance based on a result of the obtained and analyzed data, thereby having an advantage that allows a user to improve his or her body balance in daily life after the body balance correction product is worn. That is, when a body balance correction product is worn that is selected according to the body balance correction system of the present invention, pressure applied to the human joints is evenly distributed, and thus the body balance correction product is helpful in preventing and healing diseases of the joints.

In addition, the body balance correction product improves uneven activity of the human body, thereby enhancing muscular strength, and provides an optimum environment to growing kids, thereby allowing the kids to grow fast. Furthermore, when the body balance correction product is worn that is selected according to the body balance correction system of the present invention, blood flow pathways are improved and an amount of brain activity is increased, and thus the brain activity may be enhanced.

### DESCRIPTION OF DRAWINGS

FIG. 1 is a schematic block diagram of a body balance correction system according to embodiments of the present invention.

FIG. 2 illustrates an exemplary result of an examination measured after the body balance correction system according to embodiments of the present is applied to an examinee.

FIG. 3a illustrates pressure applied to the feet according to the twisted spine.

FIG. 3b illustrates a correlation between the spinal skeleton and the foot skeleton.

FIG. 3c illustrates a correlation between the human organs and the feet using left and right foot soles diagram

FIG. 3d illustrates a correlation between the human organs and the feet using the inside and outside of the foot.

FIG. 3e illustrates an exemplary result of another examination performed to the examinee so that an analysis performed on the basis of the correlations shown in FIGS. 3a to 3d is described.

FIG. 4 illustrates an example of a body balance correction product selected by the body balance correction system according to embodiments of the present invention, that is cushioning shoes, in an exploded perspective view.

FIG. 5a illustrates an example of data on a body balance state measured before the body balance correction product is worn, and FIG. 5b illustrates an example of another data on a body balance state measured after the cushioning shoes selected on the basis of a selection criterion is worn.

FIGS. 6 to 8 illustrate exemplary screen configurations of a display unit included in the body balance correction system according to embodiments of the present invention, respectively.

FIG. 9 is a flowchart of a method for controlling the body balance correction system that diagnoses and corrects a body balance state according to embodiments of the present invention.

### BEST MODE

Hereinafter, a body balance correction system and a method for controlling same according to a preferred embodiment of the present invention are described in detail with reference to the accompanying drawings.

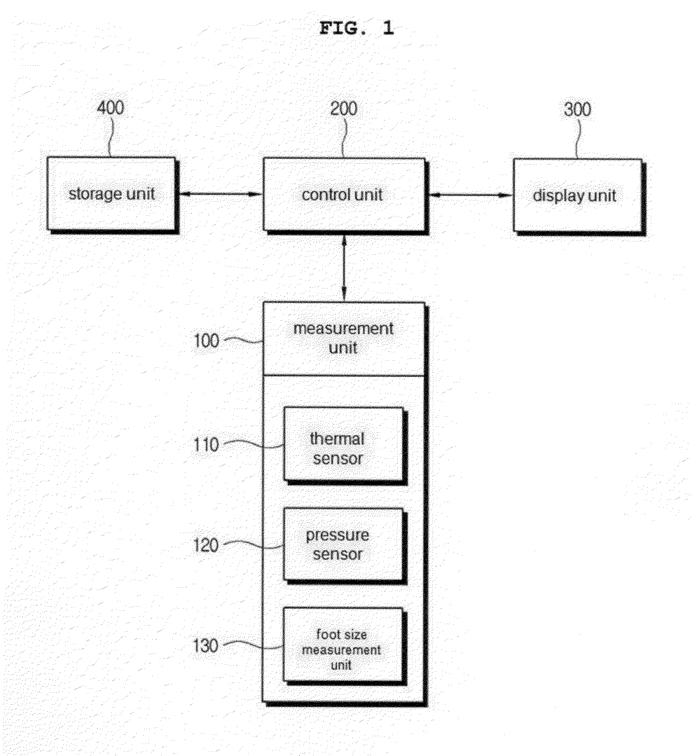

FIG. 1 is a schematic block diagram of a body balance correction system according to embodiments of the preserve invention.

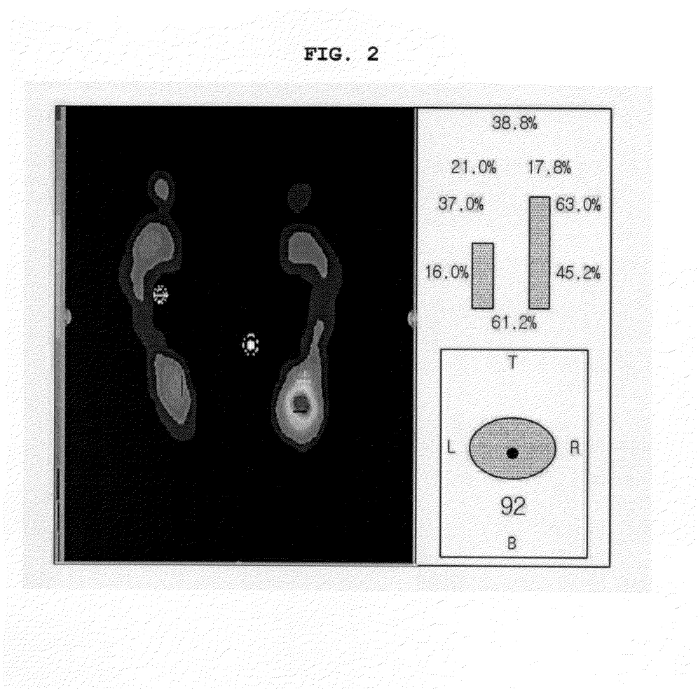

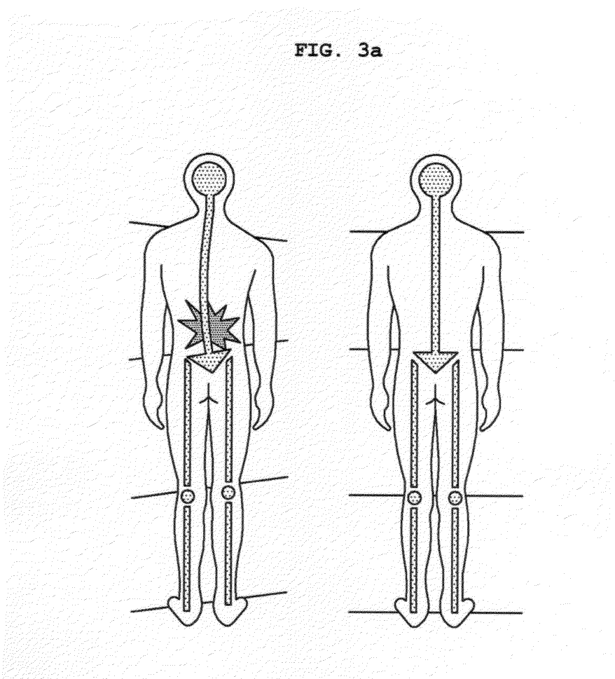

Referring to FIG. 1, the body balance correction system according to embodiments of the present invention diagnoses and corrects body balance and includes a measurement unit 100 which measures data on the left and right sides of an examinee's body, a control unit 200 which analyzes the data measured by the measurement unit 100 and provides a selection criterion for a body balance correction product based on a result of the analyzed data, and a display unit 300 which displays a result of the data analyzed by the control unit 200. The measurement unit 100 diagnoses body balance of an examinee in a state in which the examinee places his or her feet on an examination plate (not shown), and includes a thermal sensor 110 and a pressure sensor 120. The thermal sensor 110 is provided in the examination plate and senses temperatures of different parts of the examinee's left and right foot soles. The pressure sensor 120 senses body pressure of the left, right, front, and rear parts of the examinee's body. The pressure sensor 120 may have a plurality of pressure sensors which sense body pressure of the left, right, front, and rear parts of the examinee's body. In addition, the control unit 200 senses the degree by which the examinee's body is balanced, on the basis of the pressure data measured by a plurality of the pressure sensors. In addition, measurement is performed in an optimum environment because the control unit senses the degree by which the examinee wobbles and his or her body is balanced, and thus the reliability of the measurement is increased. FIG. 2 illustrates an example of an examination result displayed on the display unit after the body balance correction system according to embodiments of the present is applied to the examinee. Referring to FIG. 2, when measurement is began in a state in which the examinee places his or her feet on the examination plate, data an body balance of the examinee is obtained through a plurality of the pressures sensors 120 (Refer to the right bottom part of FIG. 2). That is, data is obtained that shows balance states of the left, right, frost, and rear parts of the examinee's body in points, and another data is obtained that shows whether the examinee wobbles at the time of measurement in percentage (member 92 in FIG. 2). Here, a higher number represents a less wobble. In addition, values obtained after the measurement is performed through a plurality of the pressure sensors, that is, results in percentage such as 37%, 63%, 38.8%, 61.2% show average pressure of the left, right, front, and rear parts of the examinee's body, respectively (Refer to the right top part or FIG. 2). Furthermore, other values in percentage shown above and below the left average pressure value in percentage, such as 21% and 16%, indicate pressure of the left front and left rear parts, respectively, and further values in percentage shown above and below the right average pressure value in percentage, such as 17.8% and 45.2%, indicate pressure of the right front and right rear parts, respectively. As described above, data on body balance is obtained on the basis of average pressure of parts of the examinee's body, and the obtained data is analyzed by the control unit 200, and thus an optimum body balance correction product is selected. In addition, temperatures of different parts of the examinee's left and right foot soles are sensed through the thermal sensors 110 (Refer to the left part of FIG. 2). FIG. 2 illustrates an example in which body temperatures measured using a plurality of the thermal sensors 110 are distinguished from each other in different colors. Here, blue represents a low temperature, and color changes from green to yellow to red as temperature becomes high. An optimum body balance correction product is selected on the basis of the measured temperatures of different parts of the examinee's foot soles and a correlation between the human organs and the feet.

In addition, the measurement unit 100 may further include a foot size measurement unit 130 for measuring the size of the examinee's foot. The foot sire measurement unit 130 measures lengths of the left and right feet and widths of the bails of the feet. Here, the foot sire measurement unit 130 adopts an electric device for automatically measuring a foot size.

Furthermore, the foot size may be manually measured using a tapeline, a thread, a straightedge, and so on. For example, the one foot having the ball wider than the other foot is measured in either of the following manners: using a tapeline; or using a thread, and then the thread is measured using a straightedge. Also, lengths of the feet are measured in such a manner that the feet are placed on a piece of paper, and then a pen is used so that two dots are put where the tips of the toes and the heel come into contact with a piece of the paper, and after that, a tapeline or straightedge is used so that the lengths of the feet are measured.

The control unit 200 analyses body data measured in the above-mentioned manner or a foot size measured in a manual manner by the measurement unit 100, and provides a selection criterion for a body balance correction product based on a result of the analyzed data or size. That is, the control unit 200 analyzes body data, based on a correlation between the foot skeleton and the spins and pelvis, another correlation between the spine and body pressure, and a further correlation between the human organs and the feet.

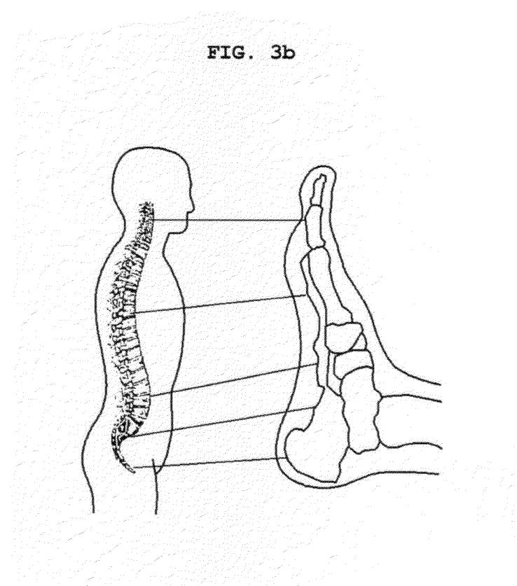

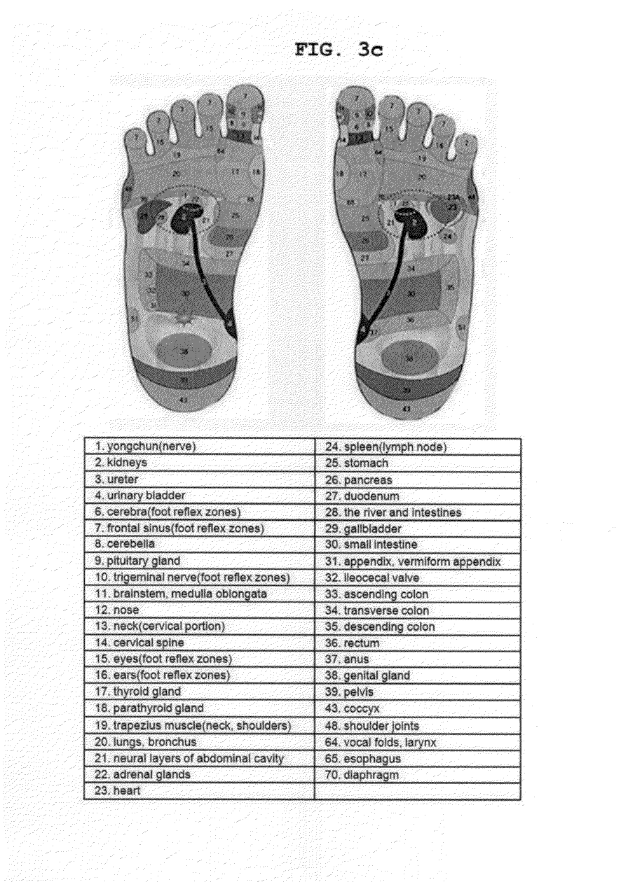

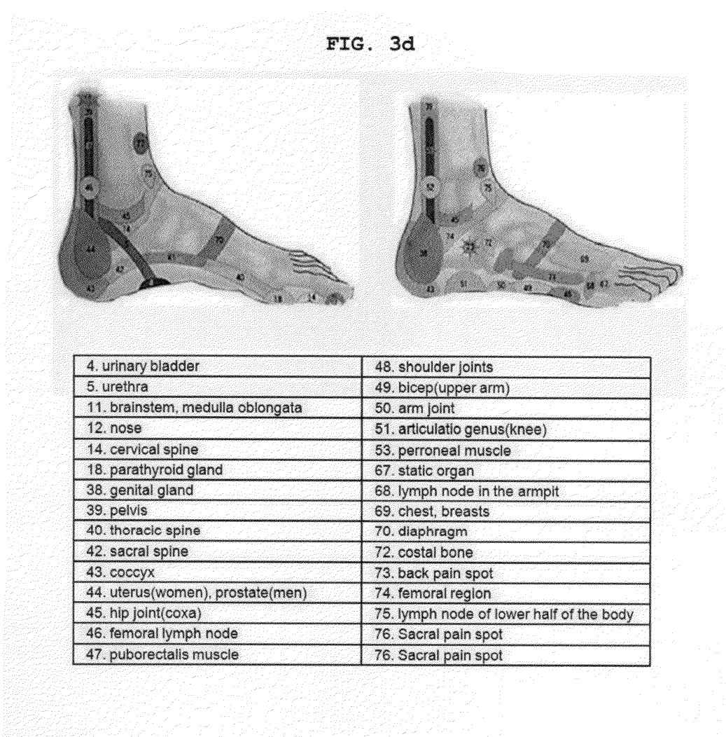

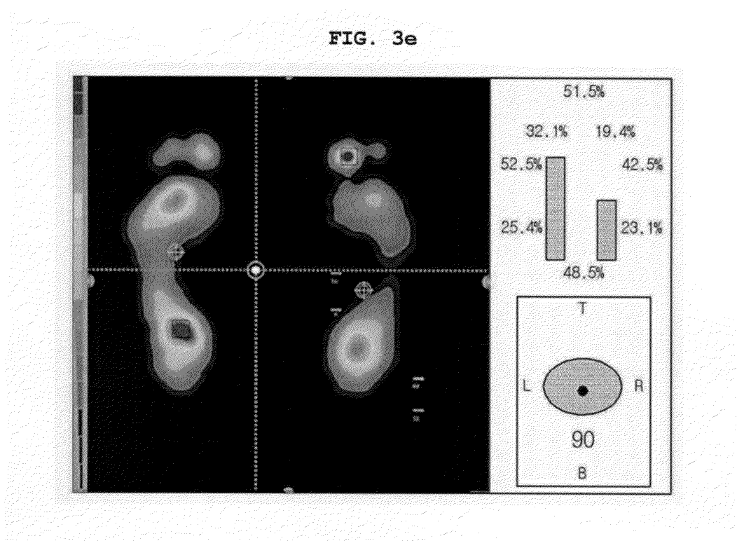

Hereinafter, an exemplary analysis of body data performed by the control unit is described with reference to FIGS. 3a to 3e.

FIG. 3a illustrates a state in which the spine is balanced and pressure applied to the feet when the spine is twisted, and FIG. 3e illustrates a result of a measurement examination so that an exemplary analysis performed using correlations shown in FIGS. 3a to 3d is described.

Referring to FIGS. 3a and 3e, the control unit 200 uses a correlation between the spine and pressure applied to the feet to analyze body balance of an examinee. That is, according to data on body pressure applied to the left and right sides of the examinee's body, which is measured by the pressure sensor 120, pressure applied to the left foot is greater than that applied to the right foot. In this case, referring to a diagram of the examinee's spine balance, body balance leans to the left side as shown in the left diagram of FIG. 3a, and thus the spine is caused to be twisted. Such an analysis is one of selection criteria for selecting a body balance correction product.

FIG. 3b illustrates a correlation between the spinal skeleton and the foot skeleton, and FIGS. 3c and 3d is a diagram of foot reflex zones showing a correlation between the human organs and the feet.

Referring to FIGS. 3b to 3d, the spinal or pelvic skeleton has a structure linked to the foot skeleton, and different parts of the feet are linked to the human organs, respectively, according to a foot reflexology.

The control unit 200 uses pressure applied to the feet and temperature data on different parts of the feet to analyze a momentum of each part of the examinee's body. That is, the spinal and pelvic skeleton is analyzed using a structure of the foot skeleton linked thereto (FIG. 3b) and diagrams of foot reflex zones (FIGS. 3c and 3d). Hereinafter, an analysis of moments of the pelvis, spine, neck, and head of the examinee is described on the basis of a result of measurement shown in FIG. 3e.

According to the analysis of the pelvic skeleton, the left part has a pressure value of 25.3% and the right part has a pressure value of 23.3%, and thus a deviation between the left and right parts is tiny. Accordingly, when compared to the diagram of foot reflex zones in FIGS. 3c and 3d, the pelvis has a tiny deviation between the left part and the right part, and thus the momentum of the pelvis is good.

According to the analysis of the spinal skeleton, the part of the foot linked to the spine is slightly raised, and thus the spine is analyzed in a twisted state by considering a correlation between the foot skeleton and the spinal skeleton. Accordingly, the spine has an unsteady momentum and works in a bad state.

According to the analysis of the neck and the head, the neck and the head are linked to the big toe in the diagram of foot reflex zones, and the part of the foot sole corresponding to the neck and the bead has a relatively-low temperature when a temperature distribution is considered, and thus the neck and the head have decreased momentia.

In addition, an amount of blood flow of the examinee is analyzed using the measurement result in FIG. 3e and the diagram of foot reflex zones.

Furthermore, the control unit 200 provides a selection criterion for a body balance correction product, based on the above-mentioned analyzed result. That is, when the body balance correction product is used during a predetermined period, the control unit 200 calculates at least one of strength, height, and a momentum adjustment value of a balance correction member configuring the body balance correction product so that the body imbalance indexes are modified to a predetermined reference value, and then provides a the selection criterion. Here, the predetermined reference value is the one in a case in which the pelvis, the spine, the neck, and the head have normal momenta in a state in which the left, right, front, and rear parts of the examinee's body are balanced.

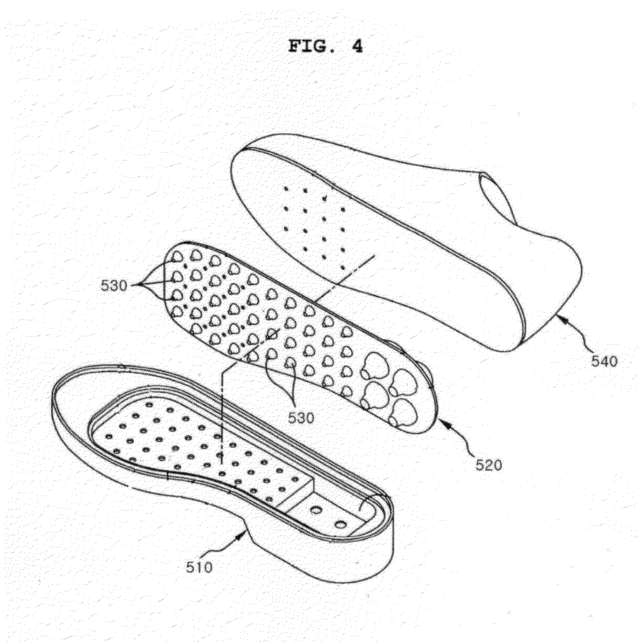

As an example of the body balance correction product, a pair of cushioning shoes may be adopted. Referring to FIG. 4, cushioning shoes 500 includes an outsole 510, a midsole 520 which is disposed on the outsole 510, a plurality of cushioning member 530, and an insole 540 which is coupled to the midsole 520 and accommodates a user's foot. A plurality of the cushioning members 530 are interposed between the midsole 520 and the outsole 510, and locally reduce impacts at the positions to which the members 530 are attached while the user walks. Here, a plurality of the cushioning members 530 are applicable to body balance correction members of the body balance correction product, and may be made in various forms according to the selection criterion provided by the control unit 200.

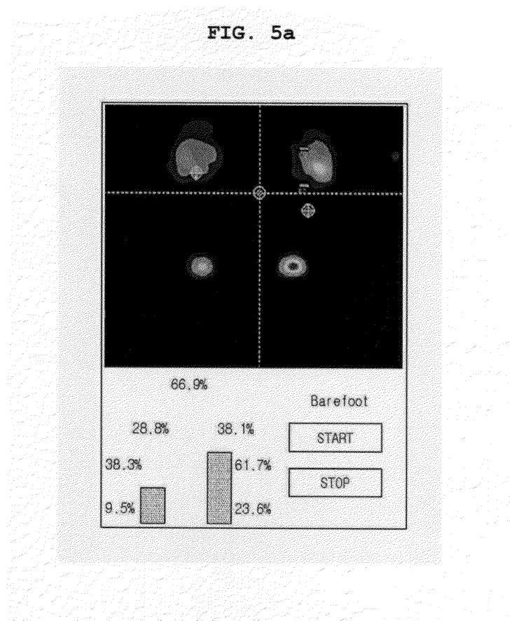

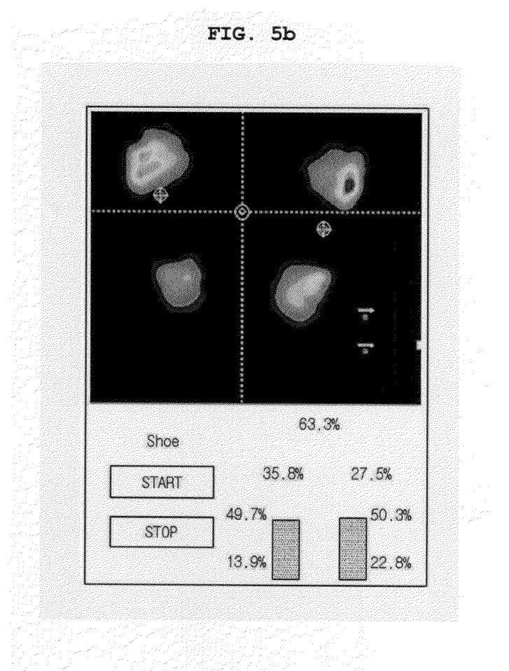

FIGS. 5a and 5b illustrate a comparison of data on body balance before and after the cushioning shoes selected on the basis of the selection criterion is worn.

FIG. 5a illustrates data on body balance measured before the cushioning shoes is worn, and FIG. 5b illustrates another data on body balance measured after the cushioning shoes selected according to the selection criterion is worn.

Referring to FIG. 5a, the left and right feet have average pressure values of 38.3% and 61.6%, respectively, and thus the pressure applied to the right foot is greater than that applied to the left foot by more than 20%.

Whereas, when body balance is measured in a state in which the cushioning shoes selected according to the selection criterion is worn, the left and right feet have average pressure values of 49.7% and 50.3%, respectively, thereby having a tiny pressure deviation, that is, a balanced state, accordingly, the body balance is remarkably improved.

The body balance correction system according to the present invention may further include a storage unit 400 which stores the body data measured by the measurement unit 100 as well as the result analyzed, by the control unit 200. That is, the storage unit 400 stores body balance indexes and momentia measured through the thermal sensor 110 and the pressure sensor 120 as well as measured data as shown in FIG. 3e. Here, the measured data may be compressed into an image file and classified by date and name. In addition, the storage unit 400 may store data related to the examinee's health in a classified manner.

The display unit 300 displays at least one of the following data on the examinee: personal information; examination date; a history of examinations; a pressure distribution on the left and right sides of the body; a temperature distribution on the left and right foot soles; a body balance state of the examinee when an examination is performed; a correlation between the foot skeleton, and the spine; a correlation between body pressure and the spine; and a correlation between the human organs and the feet.

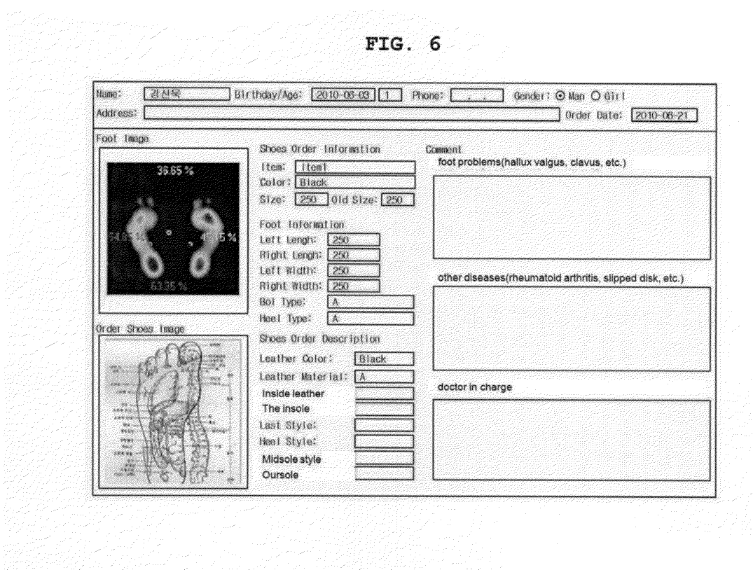

Specifically, as shown in FIG. 6, the display unit displays an analyzed result of how the human organs are distributed on foot reflex zones, based on a temperature distribution on any one of the left and right foot soles, the foot reflex zones showing a correlation between the human organs and the feet, and unusual points of the temperature distributions.

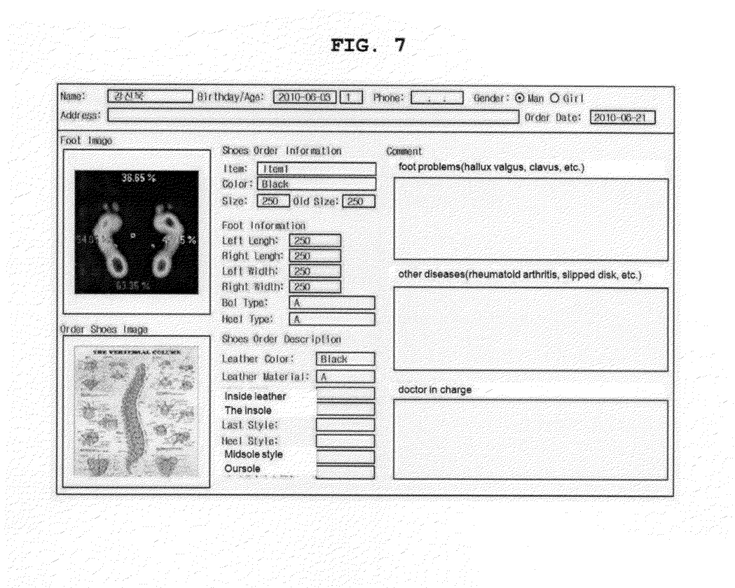

For another example, as shown in FIG. 7, the display unit may display a temperature distribution on at least one of the left and right foot soles and a spine diagram showing a correlation between the spine, the pelvis, and the feet. In addition, the display unit may display an analyzed result of twisted portions or degrees of the spine and the pelvis on the spine diagram, based on unusual points of the temperature distribution on the foot soles.

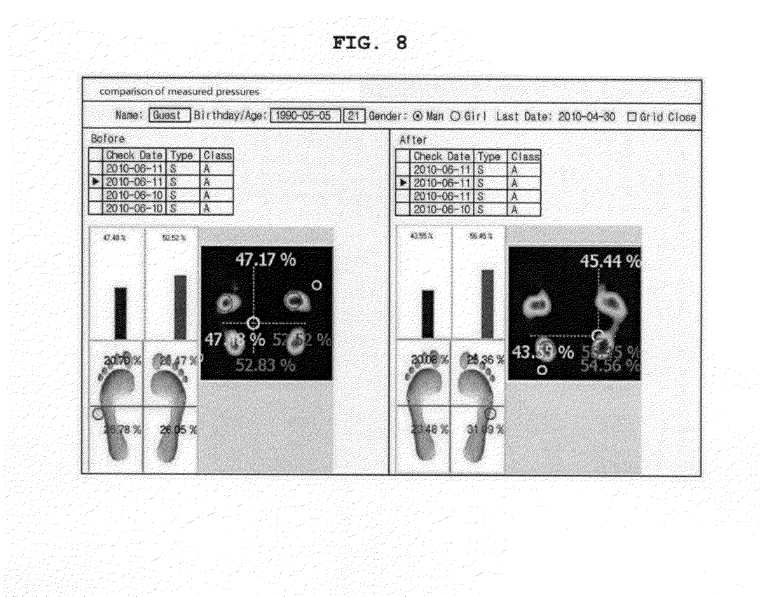

Furthermore, as shown in FIG. 8, the display unit uses a history of the examinee's examinations that are performed many times to compare and display the examination results measured at times different from each other, and thus changes in body balance of the examinee are easily checked and data on the changes are analyzed and managed.

The body balance correction system with such a configuration according to the present invention obtains body balance data of an examinee through the measurement unit 100, analyzes the body balance data through the control unit, and selects and provides a body balance correction product on the basis of an analyzed result of the body balance data, thereby having an advantage that allows the examinee to improve his or her body balance in daily life after the examinee wears the body balance correction product. In addition, the body balance correction system regularly measures and analyzes body balance data of the examinee, and then compares the measured data with other previously-recorded data, thereby having another advantage that allows the examinee to easily realize effects of his or her body balance improvement after the examinee wears the body balance correction product.

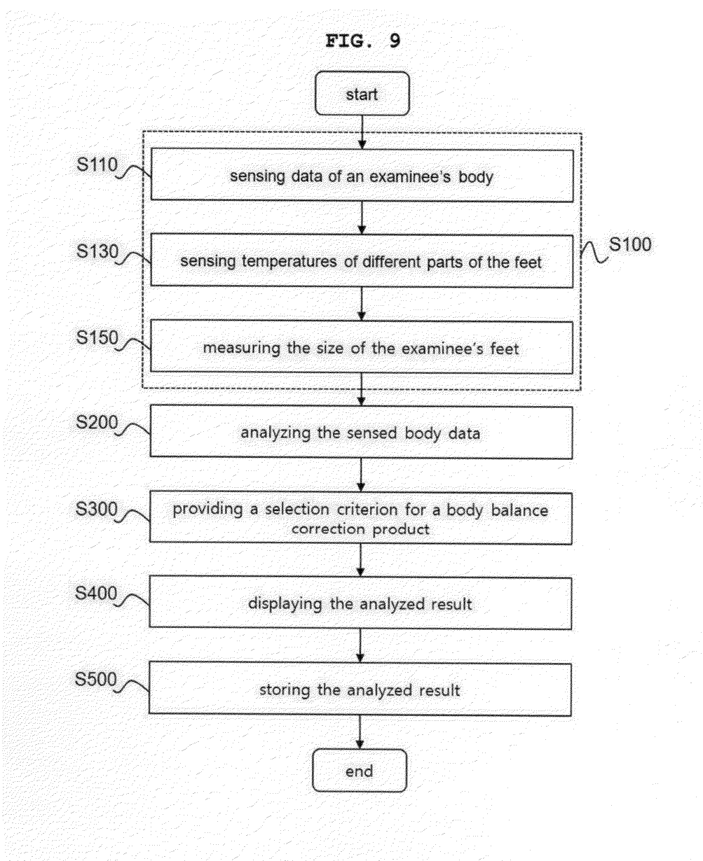

Hereinafter, a method for controlling the body balance correction system according to embodiments of the present invention is described in detail with reference to FIG. 9.

FIG. 9 is a flowchart of a method for controlling the body balance correction system that diagnoses and corrects a body balance state according to embodiments of the present invention.

Referring to FIGS. 1 and 9, the method for controlling the body balance correction system, according to embodiments of the present invention includes measuring data on the left and right sides of an examinee's body (S100), analyzing the body data measured by the measurement unit 100 (S200), providing a selection criterion for a body balance correction product on the basis of the analyzed data (S300), and displaying the analyzed, data (S400). In addition, the method for controlling the body balance correction system according to the present invention may further include storing the body data measured by the measurement unit 100 and the other data analyzed by the control unit 200 (S500).

The measuring data (S100) includes sensing body pressure of the left, right, front, and rear parts of the examinee's body using the pressure sensor 120 (S110), sensing temperatures of different parts of the examinee's left and right foot soles using the thermal sensor 110 (S130), and measuring the sire of the examinee's foot (S150).

The analyzing the body data (S200) may further include analyzing the degree by which the examinee's body is balanced. In the analyzing the body data (S200), the body data measured by the measurement unit is analyzed, on the basis of at least one of a correlation between the foot skeleton and the spine, the cervical spine, and the pelvis, another correlation between the spine and body pressure, and a further correlation between the human organs and the feet.

In the providing a selection criterion for a body balance correction product (S300), at least one of strength, height, and a momentum adjustment value of a balance correction member configuring the body balance correction product is calculated so that the analyzed result is modified according to a predetermined reference value, and then a selection criterion, is provided.

In the displaying the analyzed data (S400), at least one of data on the examinee, such as personal information, examination date, a history of examinations, a pressure distribution on the left and right sides of the body, a temperature distribution on the left and right foot soles, a body balance state of the examinee when an examination is performed, a correlation between the foot skeleton and the spine, a correlation between body pressure and the spine, and a correlation between the human organs and the feet, is displayed.

In addition, in the displaying the analyzed data (S400), a history of the examinee's examinations that are performed many times is used so that results measured at times different from each other are compared with each other and displayed.

The preferred embodiments of the invention in the foregoing description are presented to provide examples, and a variety of modifications are possible to those skilled in the art that has common knowledge of the invention. Accordingly, the protective scope of the present invention is defined by the claims below, and all the technical ideas within the scope equivalent to them come within the scope of the present invention.

1. Body balance correction system for diagnosing and correcting a body balance state, comprising:
a measurement unit which measures data on the left and right sides of an examinee's body;
a control unit which analyzes the data measured by the measurement unit and provides a selection criterion for a body balance correction product based on a result of the analyzed data; and
a display unit which displays the result of the analyzed data.

2. Body balance correction system according to claim 1, characterized in that the measurement unit comprises a pressure sensor which senses body pressure of the left, right, front, and rear parts of the examinee's body, and a thermal sensor which senses temperatures of different parts of the examinee's left and right foot soles.

3. Body balance correction system according to claim 2, characterized in that the measurement unit further comprises a foot size measurement unit for measuring the size of the examinee's foot.

4. Body balance correction system according to claim 2, characterized in that the pressure sensor has a plurality of pressure sensors which sense body pressure of the left, right, front, and rear parts of the examinee's body, and the control unit senses the degree by which the examinee's body is balanced, on the basis of the body pressure data measured by a plurality of the pressure sensors.

5. Body balance correction system according to claim 1, characterized in that the control unit analyzes the body data measured by the measurement unit, based on at least one of a correlation between the foot skeleton and the spine, the cervical spine, and the pelvis, another correlation between the spine and body pressure, and a further correlation between the human organs and the feet.

6. Body balance correction system according to claim 5, characterized in that the control unit calculates at least one of strength, height, and a momentum adjustment value of a balance correction member configuring the body balance correction product so that the analyzed result is modified according to a predetermined reference value, and then provides a selection criterion.

7. Body balance correction system according to claim 1, characterized in that the system further comprises a storage unit which stores the data measured by the measurement unit as well as the result analyzed by the control unit.

8. Body balance correction system according to claim 7, characterized in that the display unit displays at least one of the following data on the examinee: personal information; examination date; a history of examinations; a pressure distribution on the left and right sides of the body; a temperature distribution on the left and right foot soles; a body balance state of the examinee when an examination is performed; a correlation between the foot skeleton and the spine; a correlation between body pressure and the spine; and a correlation between the human organs and the feet.

9. Body balance correction system according to claim 8, characterized in that the display unit displays an analyzed result of how the human organs are distributed on foot reflex zones, based on a temperature distribution on any one of the left and right foot soles, the foot reflex zones showing a correlation between the human organs and the feet, and unusual points of the temperature distribution.

10. Body balance correction system according to claim 8, characterized in that the display unit uses data of a temperature distribution on at least one of the left and right foot soles, a spine diagram showing a correlation between the spine, the pelvis, and the feet, and unusual points the temperature distribution in order to display an analyzed result of twisted portions or degrees of the spine and the pelvis on the spine diagram.

11. Body balance correction system according to claim 8, characterized in that the display unit uses a history of the examinee's examinations that are performed many times in order to compare and display the examination results measured at times different from each other.

12. Body balance correction system according to claim 1, characterized in that the body balance correction product is a pair of cushioning shoes which comprises an outsole, a midsole that is disposed over the outsole, a plurality of cushioning members that are interposed between the midsole and the outsole to locally reduce impacts, and an insole that is coupled to the midsole and accommodates a user's foot.

13. Method for controlling a body balance correction system that diagnoses and corrects body balance, comprising: measuring data on the left and right sides of an examinee's body; analyzing the data measured by the measurement unit; providing a selection criterion for a body balance correction product, based on a result of the analyzed data; and displaying the result of the analyzed data.

14. Method for controlling a body balance correction system according to claim 13, characterized in that the measuring data on the left and right sides of the examinee's body comprises sensing body pressure of the left, right, front, and rear parts of the examinee's body, sensing temperatures of different parts of the examinee's left and right foot soles, and measuring the size of the examinee's foot.

15. Method for controlling a body balance correction system according to claim 14, characterized in that the analyzing the data measured by the measurement unit further comprises analyzing the degree by which the examinee's body is balanced when measuring data on the examinee's body is performed.

16. Method for controlling a body balance correction system according to claim 13, characterized in that, in the analyzing the data measured by the measurement unit, the body data is analyzed, based on at least one of a correlation between the foot skeleton and the spine, the cervical spine, and the pelvis, another correlation between the spine and body pressure, and a further correlation between the human organs and the feet.

17. Method for controlling a body balance correction system according to claim 16, characterized in that, in the providing a selection criterion for a body balance correction product, at least one of strength, height, and a momentum adjustment value of a balance correction member configuring the body balance correction product is calculated so that the analyzed result is modified according to a predetermined reference value, and then a selection criterion is provided.

18. Method for controlling a body balance correction system according to claim 13, characterized in that the method for controlling a body balance correction system further comprises storing the data measured by the measurement unit as well as the result analyzed by the control unit.

19. Method for controlling a body balance correction system according to claim 18, characterized in that, in the displaying the result of the analyzed data, at least one of the following data on the examinee: personal information; examination date; a history of examinations; a pressure distribution on the left and right sides of the body; a temperature distribution on the left and right foot soles; a body balance state of the examinee when an examination is performed; a correlation between the foot skeleton and the spine; a correlation between body pressure and the spine; and a correlation between the human organs and the feet is displayed.

20. Method for controlling a body balance correction system according to claim 19, characterized in that, in the displaying the result of the analyzed data, examination results measured at times different from each other are compared with each other and displayed on the basis of a history of the examinee's examinations that are performed many times.
